(19) United States
(12) Patent Application Publication
(10) Pub. No.: US 2019/0298311 A1
(43) Pub. Date: Oct. 3, 2019

(54) INTRAVASCULAR FLOW DETERMINATION

(71) Applicant: KONINKLIJKE PHILIPS N.V., EINDHOVEN (NL)

(72) Inventors: Christian HAASE, Hamburg (DE); Michael GRASS, Buchholz in der Nordheide (DE)

(73) Assignee: KONINKLIJKE PHILIPS N.V., EINDHOVEN (NL)

(21) Appl. No.: 16/339,159

(22) PCT Filed: Sep. 27, 2017
(86) PCT No.: PCT/EP2017/074426

§ 371 Date: Apr. 3, 2019

(30) Foreign Application Priority Data
Oct. 7, 2016 (EP) 16192768.6

(51) Int. Cl. A61B 8/08; A61B 6/12; A61B 8/06; A61B 8/12; A61B 5/0215; A61B 6/00; A61B 8/00

(52) U.S. Cl. CPC A61B 8/5261; A61B 6/12; A61B 8/06; A61B 8/12; A61B 8/4416; A61B 5/0215; A61B 6/481; A61B 6/504; A61B 8/5223; A61B 8/488

(57) ABSTRACT

The present invention relates to intravascular flow determination. In order to provide a facilitated way to determine flow values with improved accuracy, an intravascular flow determination device (50) is provided that comprises an input unit (54), a data processing unit (52), and an output unit (56). The input unit is configured to provide a measured local flow velocity value of a fluid inside a vessel of an object, which local flow velocity value is measured at a local position of interest, and to provide local spatial data of the vessel and the local position of interest; wherein the local flow velocity value, and the local spatial data relate to the same in position in time; and to provide a model flow-profile. The data processing unit is configured to adapt the model flow-profile based on the local values and the spatial data of the vessel and fluid dynamic constraints to generate an adapted local flow-profile relating to a cross-section at the local position of interest; and to determine a local peak flow value of the fluid inside the vessel based on the generated adapted local flow-profile. The output unit is configured to provide the local peak flow value.

### FIELD OF THE INVENTION

The present invention relates to intravascular flow determination, and relates in particular to an intravascular flow determination device, to a hemodynamic system for intravascular flow determination and to a method for determining intravascular flow.

### BACKGROUND OF THE INVENTION

Blood flow measurements may be used for example in cardiology to quantify the severity of coronary stenosis. Among others, the most widely used approach today is the use of flow sensing catheters. The flow is measured using a forward looking ultrasound sensor inserted in the vessel. U.S. Pat. No. 6,601,459 B1 relates to a method of volumetric blood flow measurement. However, it is sometimes cumbersome to achieve a stable positioning for the measuring.

### SUMMARY OF THE INVENTION

There may be a need to provide a facilitated way to determine flow values with improved accuracy.

The object of the present invention is solved by the subject-matter of the independent claims; further embodiments are incorporated in the dependent claims. It should be noted that the following described aspects of the invention apply also for the intravascular flow determination device system, and for the hemodynamic system for intravascular flow determination and for the method for determining intravascular flow as well as to for the computer program element for controlling an apparatus and for the computer readable medium.

According to the present invention, an intravascular flow determination device is provided that comprises an input unit, a data processing, and an output unit. The input unit is configured to provide a local flow velocity value of a fluid measured with a flow sensor inside a vessel of an object, which local flow velocity value is measured at a local position of interest, and to provide local spatial data of the vessel and the local position of interest. The local flow velocity value, the local spatial data relate to the same position in time; and to provide a model flow-profile. The data processing unit is configured to adapt the model flow-profile based on the local values and the spatial data of the vessel and fluid dynamic constraints to generate an adapted local flow-profile relating to a cross-section at the local position of interest; and to determine a local peak flow value of the fluid inside the vessel based on the generated adapted local flow-profile. Further, the output unit is configured to provide the local peak flow value.

Due to adapting a flow model based on current data, considering the spatial arrangement within the vessel, a careful and stable positioning is no longer necessary to measure the flow in the coronary artery.

To measure flow, so called flow-wire (or a combo wire with an additional pressure sensor) can be used.

It is noted that the terms “input unit” and “output unit” relate to the data exchange to and from the data processing unit. The input unit and output unit can be provided as an integral part of a processor forming the data processing unit or as distinct elements. The input unit and output unit can also be provided as a combined interface providing data exchange in both ways, integrally formed or distinct.

The term “to provide the local peak flow value” relates to further use of the value, e.g. for further processing or for being used for displaying information.

In an example, the data processing unit is configured to receive a measured local flow velocity value of a fluid inside a vessel of an object, which local flow velocity value is measured at a local position of interest, and to receive local spatial data of the vessel and the local position of interest. The local flow velocity value, the local spatial data relate to the same position in time; and to receive a model flow-profile. The data processing unit is configured to adapt the model flow-profile based on the local values and the spatial data of the vessel and fluid dynamic constraints to generate an adapted local flow-profile relating to a cross-section at the local position of interest; and to determine a local peak flow value of the fluid inside the vessel based on the generated adapted local flow-profile. Further, the data processing unit is configured to output the local peak flow value.

In an example, a display or graphical user interface may be provided to indicate the local peak flow value, e.g. as value (numbers) or graph or other graphic illustration.

According to an example, the input unit is further configured to provide a local pressure value of the fluid inside the vessel for the local position of interest. The local pressure value relates to the same position in time. The data processing unit is configured to adapt the model flow-profile also based on the local pressure value.

According to an example, the data processing unit is configured to output a ratio of two local peak flow velocities at two distinct locations. A first location is distal to a second location in the vessel.

According to the present invention, also a hemodynamic system for intravascular flow determination is provided. The system comprises an X-ray imaging device, a flow measure device comprising the flow sensor; and an intravascular flow determination device according to one of the preceding examples. The flow measure device is configured to be arranged inside a vessel and to measure the local flow velocity value. The X-ray imaging device comprises an X-ray source and an X-ray detector to acquire image data of a region of interest of the vessel comprising a local position of interest. The data processing unit is configured to determine a position of the flow measure device arranged inside the vessel based on the acquired image data.

According to an example, it is further provided a pressure detection device. The pressure detection device is configured to detect a local pressure value; and the data processing unit is configured to determine a position of the pressure detection device arranged inside the vessel based on the acquired image data.

According to an example, for adapting the model flow-profile, the data processing unit is configured to provide fluid dynamic constraints that comprise at least one of the following: physiological data of the patient, such as age, weight, blood viscosity or other blood values, or a local pressure value, vessel diameter derived from the spatial data, vessel position derived from the spatial data, relative position of the flow measure device within the vessel, measured blood flow velocity at the position of the flow measure device, and analytic equations based on a tube with a friction coefficient. According to an example, the flow measure device is an ultrasound device; and the flow is measured with Doppler ultrasound in a viewing direction.

According to the present invention, also a method for determining intravascular flow is provided. The method comprises the following steps:
a) providing a local flow velocity value of a fluid measured with a flow sensor inside a vessel of an object, which local flow velocity value is measured at a local position of interest;
b) providing local spatial data of the vessel and the local position of interest; wherein the local flow velocity value and the local spatial data relate to the same position in time;
c) providing a model flow-profile;
d) adapting the model flow-profile based on the local values and the spatial data of the vessel and fluid dynamic constraints to generate an adapted local flow-profile relating to a cross-section at the local position of interest;
e) determining, based on the generated adapted local flow-profile:
   i) a local peak flow value of the fluid inside the vessel; and/or
   ii) a local value for volumetric flow rate.

In an example, it is provided a step al) of providing a local pressure value of the fluid inside the vessel for the local position of interest; the local pressure value relates to the same position in time; and wherein in step d) the adapting of the model flow-profile is also based on the local pressure values.

In an example, for b) it is provided: generating at least one angiogram, for which at least one angiogram contrast agent injected X-ray images are acquired.

In an example, the fluid dynamic constraints comprise at least one of the following: physiological data of the patient, such as age, weight, blood viscosity or other blood values, or a local pressure value; vessel diameter derived from the spatial data;—vessel position derived from the spatial data; relative position of the flow measure device within the vessel; measured blood flow velocity at the position of the flow measure device; and analytic equations based on a tube with a friction coefficient.

In an example, in e), the adapted local flow-profile is determined by using a finite element fluid dynamics model as fluid dynamic constraints, wherein the finite element fluid dynamics model has as input parameters a local vessel geometry including a radius of the vessel, a relative position of the flow measure device within the vessel and the measured blood flow velocity at the position of the flow measure device.

In an example, the ultrasound device has field of view and images an area displaced in the viewing direction; wherein for the local spatial data, a position of the ultrasound device is detected; and wherein a displacement factor is applied for transforming the detected position of the ultrasound device into location data of the field of view in order to use the location data of the field of view as the local spatial data.

According to an aspect, an integration of intravascular pressure and flow measurements with a hemodynamic simulation is provided based on a vascular model generated from angiography to determine not only the absolute flow level in a vessel but also the flow-profile. Additionally, the robustness of the measured flow value is improved by reducing the dependence of the flow measurement on the wire positioning.

In an example, angiography projections of the target vessel are acquired in combination with intravascular flow and pressure measurements. Further, a 3D vascular model is generated from an angiography projection and a hemodynamic simulation is performed using the measured pressure data as boundary conditions. The hemodynamic parameters predicted from the fluid dynamics simulation and the measured pressure and flow values are combined to derive additional quantities of interest.

As an advantage, the position and/or orientation of the sensor within the vessel can vary, since the actual position is detected and considered for the adaptation of the flow-profile. Hence, deviations, whether small or large, of the position and/or orientation of the sensor within the vessel do no longer lead to inaccurate flow information of the current situation. As a result, reliable flow velocity assessment is provided. Thus, true flow velocity for a cross section of the vessel can be derived for any particular relative position and orientation of the flow-wire within the vessel, resulting in faster measurements with improved accuracy.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the following drawings:

FIG. 1 illustrates a coronary vessel with a combo wire measurement resulting in a flow-profile calculated at the position of the flow sensor.

FIG. 2 schematically shows an intravascular flow determination device.

FIG. 3 shows a hemodynamic system for intravascular flow determination with an X-ray imaging device, a flow measure device and an example of the intravascular flow determination device of FIG. 2.

FIG. 4 illustrates two possible flow-profiles. The left figure shows a steep profile and the right shows a flat profile.

FIG. 5 illustrates a coronary vessel segment with a flow sensing probe. The probe position on the left is centered and allows to measure the peak flow velocity. The probe position on the right provides the flow measurement from a different part of the flow-profile.

FIG. 6 shows basic steps of an example of a method for determining intravascular flow.

### DETAILED DESCRIPTION OF EMBODIMENTS

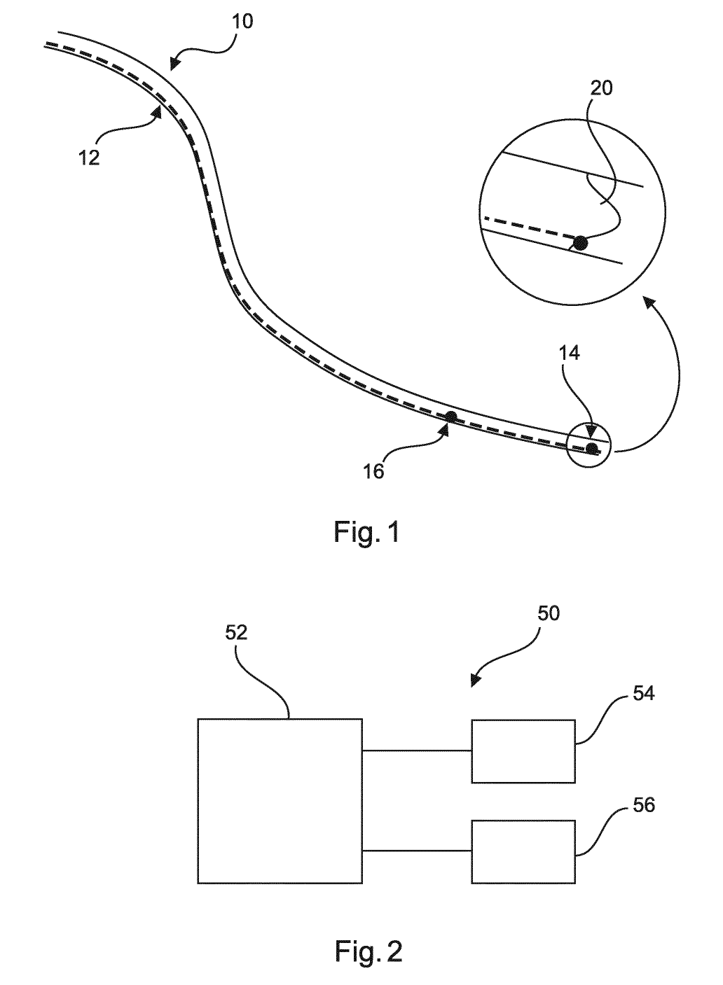

FIG. 1 shows a schematic illustration of a vessel 10, for example, of a patient. A wire 12 is inserted in the vessel for measuring purposes. The wire is provided with a flow sensor 14 at a distal end, and, as an option, with a pressure sensor 16, also on the distal end or along the wire. In the right part, a circle is showing an enlargement of the situation around the distal end. A blood flow-profile 20 is indicated, which will be described below in more detail.

FIG. 2 shows an intravascular flow determination device 50, comprising a data processing unit 52. Further, an input unit 54, and an output unit 56 is provided. The input unit 54 is configured to provide a measured local flow velocity value of a fluid inside a vessel of an object, which local flow velocity value is measured at a local position of interest, and to provide local spatial data of the vessel and the local position of interest. The local flow velocity value, and the local spatial data relate to the same moment in time. The input unit 54 is configured to provide a model flow-profile. The data processing unit 52 is configured to adapt the model flow-profile based on the local values and the spatial data of the vessel and fluid dynamic constraints to generate an adapted local flow-profile relating to a cross-section at the local position of interest. The data processing unit 52 is also configured to determine a local peak flow value of the fluid inside the vessel based on the generated adapted local flow-profile. The output unit 56 is configured to provide the local peak flow value.

In an example, not shown in detail, the input unit 54 is further configured to provide a local pressure value of the fluid inside the vessel for the local position of interest. The local pressure value relates to the same moment in time. The data processing unit 52 is configured to adapt the model flow-profile also based on the local pressure value.

In an example, not shown in detail, the data processing unit 52 is configured to output a ratio of two local peak flow velocities at two distinct locations. A first location is distal to a second location in the vessel. As a result, a ratio of two flow values is provided, V_{distal}/Vₚᵣₒₓᵢₘₐₗ, as a so-to-speak alternative option to fractional flow reserve determined as a ratio of distal pressure and proximal pressure measured in a vessel.

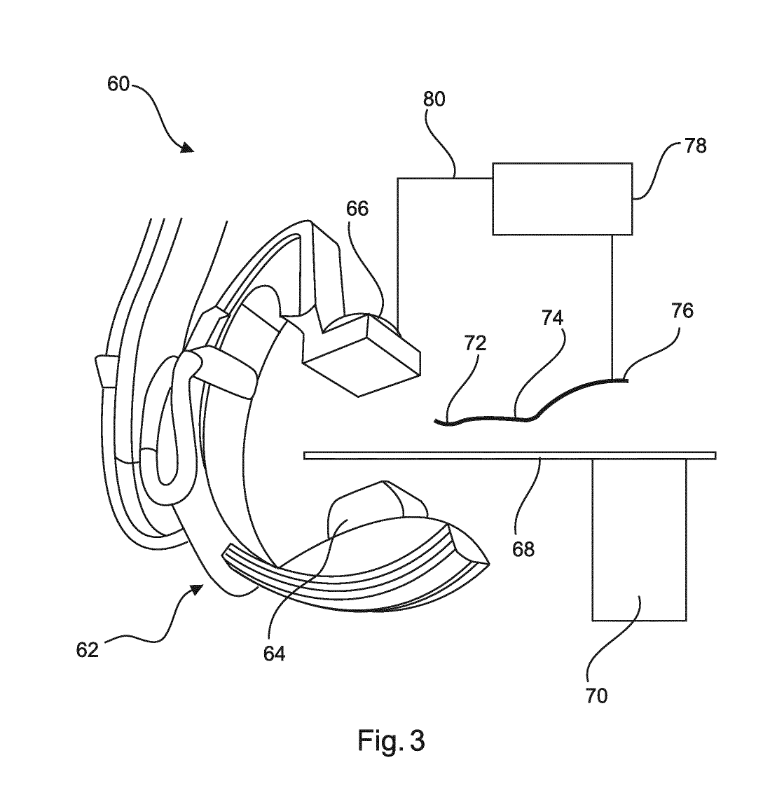

FIG. 3 shows a hemodynamic system 60 for intravascular flow determination. The system 60 comprises an X-ray imaging device 62. The X-ray imaging device 62 is indicated with an X-ray source 64 and an X-ray detector 66 to acquire image data of a region of interest of the vessel comprising a local position of interest, wherein the C-arch is only an example. Other types of mobile and stationary X-ray imagers are also provided. An object support, e.g. a patient table 68 is indicated, supported by an adaptable stand 70.

Further, a flow measure device 72 is provided. As an option, it is further provided a pressure detection device 74. For example, the flow measure device 72 and the pressure detection device 74 are provided along a wire 76 to be inserted into a body.

Further, an example of the intravascular flow determination device 78 is provided. The flow measure device 72 is configured to be arranged inside a vessel and to measure a local flow value. The data processing unit 52 is configured to determine a position of the flow measure device 72 arranged inside the vessel based on the acquired image data.

The pressure detection device 74 is configured to detect a local pressure value; and the data processing unit is configured to determine a position of the pressure detection device 74 arranged inside the vessel based on the acquired image data.

In an example, for adapting the model flow-profile, the data processing unit 52 is configured to provide fluid dynamic constraints that comprise at least one of the following: physiological data of the patient, such as age, weight, blood viscosity or other blood values, or a local pressure value, a vessel diameter derived from the spatial data, a vessel position derived from the spatial data, relative position of the flow measure device within the vessel, measured blood flow velocity at the position of the flow measure device, and analytic equations based on a tube with a friction coefficient.

In an example, not shown, for adapting the model flow-profile, the data processing unit is configured to use a finite element fluid dynamics model as fluid dynamic constraints, wherein the finite element fluid dynamics model has as input parameters a local vessel geometry including a radius of the vessel, a relative position of the flow measure device within the vessel and the measured blood flow velocity at the position of the flow measure device, and, preferably, the measured local pressure value.

The flow measure device is an ultrasound device; and, preferably, the flow is measured with Doppler ultrasound in a viewing direction.

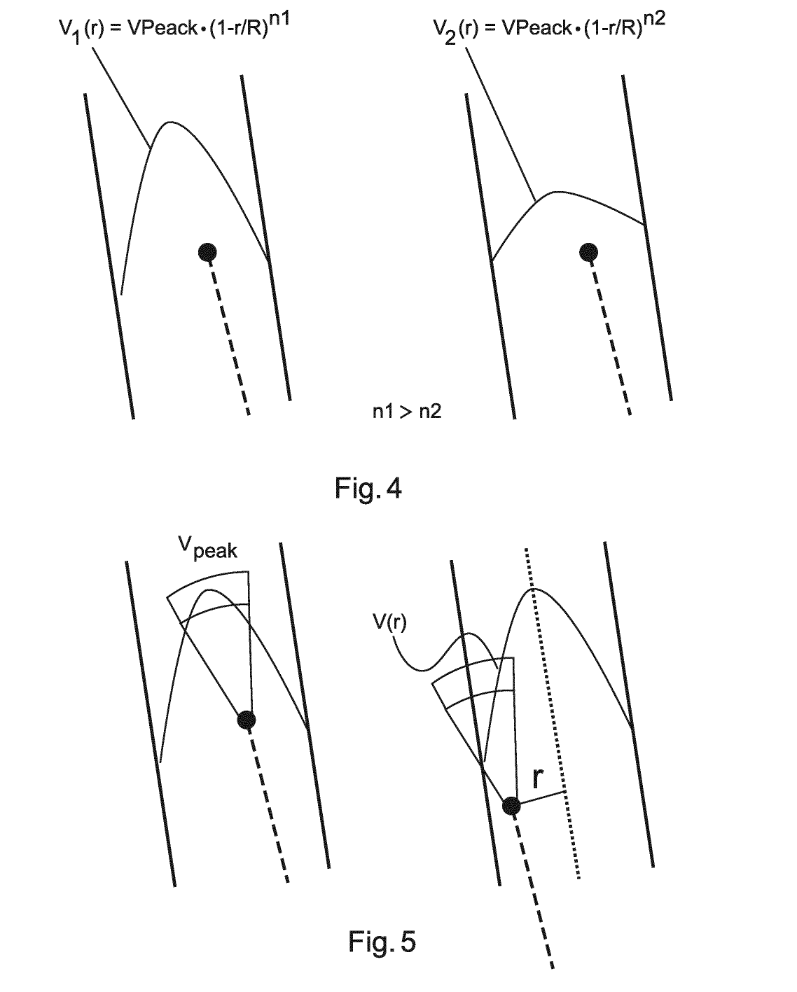

FIG. 4 illustrates two possible flow-profiles. The left figure shows a steep profile and the right shows a flat profile. By determining the spatial situation, e.g. via X-ray images, it is possible to graphically insert a model flow-profile into the vessel. Depending on the fluid-dynamic constraints, the flow-profile is adapted. For example, the flow-profile is adapted to be a steep profile or a flat profile. The current flow value is measured at the indicated location of the flow measure device 72. Since this point can be indicated in relation to the flow-profile, it is now possible to determine the peak flow value on the flow-profile.

FIG. 5 illustrates a coronary vessel segment with a flow sensing probe. The probe position on the left is centered and allows to measure the peak flow velocity. The probe position on the right provides the flow measurement from a different part of the flow-profile. With reference also to FIG. 1, for example, the clinical application is facilitated, as an orienting and/or positioning of the sensor co-axial with the axis of the vessel is not essential for achieving a reliable flow assessment. Even if the orientation of the sensor is not in the direction along the axis of the vessel and the position of the sensor is not coaxial, due to detecting the spatial situation via e.g. X-ray imaging and considering this for the adaptation of the flow-profile, an accurate result can be achieved. This means relief in clinical practice.

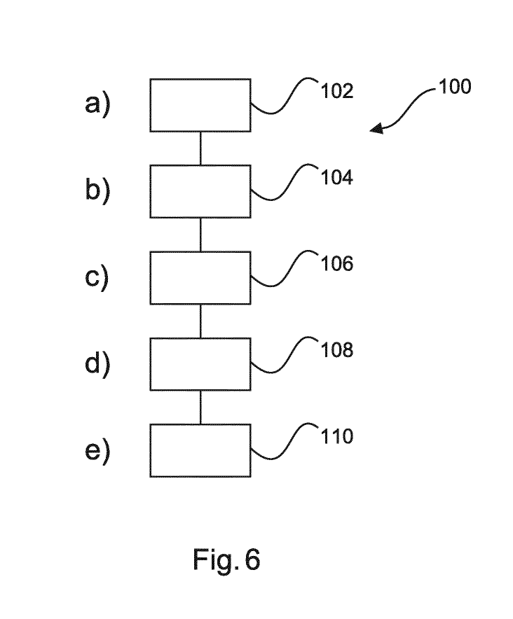

FIG. 6 shows a method 100 for determining intravascular flow, comprising the following steps: In a first step 102, also referred to as step a), a measured local flow velocity value of a fluid inside a vessel of an object is provided, which local flow velocity value is measured at a local position of interest. In a second step 104, also referred to as step b), local spatial data of the vessel and the local position of interest are provided. The local flow velocity value and the local spatial data relate to the same position in time. Further, in a third step 106, also referred to as step c) a model flow-profile is provided. In a fourth step 108, also referred to as step d), the model flow-profile is provided based on the local values and the spatial data of the vessel and fluid dynamic constraints to generate an adapted local flow-profile relating to a cross-section at the local position of interest. In a fifth step 110, also referred to a step e), based on the generated adapted local flow-profile, i) a local peak flow value of the fluid inside the vessel is determined; and/or ii) a local value for volumetric flow rate is determined.

Preferably, it is provided a step al) of providing a local pressure value of the fluid inside the vessel for the local position of interest; wherein the local pressure value relates to the same position in time; and wherein in step d) the adapting of the model flow-profile is also based on the local pressure values.

In an example, a ratio of two local peak flow velocities at two distinct locations is provided, wherein a first location is distal to a second location in the vessel. As a result, a ratio of two flow values V_{distal}/Vₚᵣₒₓᵢₘₐₗ is provided. In order to obtain peak flow velocity ratios along a segment of a vessel, the medical instrument, such as a flow-wire, may be pulled through the vessel, thereby allowing subsequent measurements of flow velocities along the vessel at respective locations, for which the peak flow velocities are ascertained. Alternatively, the medical instrument may comprise multiple flow sensors along its length.

The object may be a patient.

The position of interest can also be referred to as point of interest.

The derived adapted local flow-profile is provided across the vessel.

The measure of the local flow velocity value is also referred to as an instant flow measurement.

The local flow velocity value is a measured flow velocity value.

The local peak flow value is a determined peak flow value. Due to the adapting, the local peak flow value can also be referred to as corrected peak flow value.

The determined local peak flow value is also referred to as true peak flow velocity.

In another example, for a) it is provided: measuring the local flow value at the local position of interest with a flow measure device arranged inside the vessel; wherein for b) it is provided: measuring the local pressure value with a pressure device arranged inside the vessel; and wherein for c) it is provided: acquiring image data of a region of interest of the vessel comprising the local position of interest; and generating the local spatial data based on the image data; and determining a position of the flow measure device arranged inside the vessel based on the acquired image data.

In an example, the flow measure device and the pressure device are provided as an integrated flow measure device measuring both parameters.

The flow measure device for measuring the local flow value is also referred to as flow-wire.

In an example, for c) it is provided: acquiring at least one X-ray image.

In an option, for c) it is provided:
determining a position of the pressure detection device arranged inside the vessel based on the acquired image data.

In an example, it is provided that, wherein for c) it is provided: generating at least one angiogram, for which at least one angiogram contrast agent injected X-ray images are acquired.

In an example, instead of a current angiogram, 3D object data is provided that is based on previously acquired date, and the 3D object data is mapped with/aligned to/or registered with the current spatial situation of the vessel, i.e. the patient. Therefore, the current spatial situation is detected. For example, a 2D X-ray image is acquired. The spatial situation of the object can also be detected by position markers temporarily attached to the object.

In an example, the position of the pressure device arranged inside the vessel is derived from an electromagnetic position marker detecting arrangement. For example, the pressure device comprises at least one marker and the position of the marker is detected from sensors arranged in the vicinity.

In an example, the fluid dynamic constraints comprise at least one of the following: physiological data of the patient, such as age, weight, blood viscosity or other blood values, or a local pressure value, vessel diameter derived from the spatial data, vessel position derived from the spatial data, relative position of the flow measure device within the vessel, measured blood flow velocity at the position of the flow measure device; and analytic equations based on a tube with a friction coefficient.

The adapted local flow-profile is also referred to as adapted flow-profile. As the flow-profile indicates flow velocity across the vessel, the adapted local flow-profile can be provided as a flow-velocity-profile.

In an example, in e), the adapted local flow-profile is determined by using a finite element fluid dynamics model as fluid dynamic constraints, wherein the finite element fluid dynamics model has as input parameters a local vessel geometry including a radius of the vessel, a relative position of the flow measure device within the vessel and the measured blood flow velocity at the position of the flow measure device.

In an option, in e), the adapted local flow-profile is determined, also based on the detected pressure at the position of the pressure detection device.

Once the flow-velocity-profile for the cross section is known, the true peak flow velocity is derived.

Vessel geometry and the relative position of the flow-wire within the vessel are derived from at least one angiographic projection.

The fluid dynamic constraints relate to the flow-profile in order to modify a model flow-profile such that a modified local flow-profile is provided. The fluid dynamic constraints relate to finite elements modelling. The fluid dynamic constraints can also be referred to as hemodynamic constraints.

In an example, the ultrasound device has field of view and images an area displaced in the viewing direction; for the local spatial data, a position of the ultrasound device is detected; and wherein a displacement factor is applied for transforming the detected position of the ultrasound device into location data of the field of view in order to use the location data of the field of view as the local spatial data.

In another example, the average volume flow is predicted by the hemodynamic simulation and the locally measured peak flow velocity are used to determine the shape of the local flow-profile within the vessel.

Finite element numerical fluid dynamics, lumped model fluid dynamics, or other approaches that facilitate the simulation of fluid dynamic systems can be used to predict the absolute flow in a vessel based on a pressure gradient measurement. In a simple implementation, a resistance term R can be calculated for a given vessel geometry. The volume flow Q can then be calculated from a pressure gradient Δp using: Q=Δp/R. The average flow velocity V_{A} at a given location follows directly using the known cross sectional area A of the vessel at that location: V_{A}=Q/A. A simple flow-profile V(r) depending on the distance r from the vessel center can be predicted using the analytical expression: V(r)=V_{Peak}·(1−r/R)⁽ⁿ⁾. Where R is the radius of the vessel. The exponent n may vary depending e.g. on fluid dynamic parameters like the roughness of the vessel wall or the viscosity of the fluid. Lower values of n correspond to a flat profile and high values to a steep profile (see also FIG. 2). Based on the wire based measurement of V_{Peak} and the simulation based, average flow value, the shape of the profile can be determined: n=V_{Peak}/V_{A}−1.

If the wire position and orientation are determined from the angiography projection, also different parts of the flow-profile may be measured than only V_{Peak}. To determine the shape of a more complex flow-profile, multiple measurements at multiple positions may be taken.

In another example, for example, in case the wire positioning is uncertain, the measured peak flow may not represent the true peak flow velocity. The shape of the flow-profile can be determined by e.g. a finite element fluid dynamics modeling. The angiography data can be used to determine the measurement position r relative to the flow-profile. This allows to estimate which part V(r) of the flow-profile is evaluated by the wire. Based on the shape of the simulated flow-profile the peak flow velocity can be robustly determined independent on the wire position (see also FIG. 3): V_{Peak}=V(r)/(1−r/R)⁽ⁿ⁾.

In another example, based on a simulated flow-profile (using e.g. finite element fluid dynamics modeling and the measured pressure gradient as boundary condition) and the measured flow velocity V(r), the measurement position r of the wire relative to the flow-profile may be determined (see also FIG. 3): r=R(1−(V(r)/V_{Peak})^{(1/n)}).

In another example, some boundary conditions for fluid dynamics modeling may be uncertain like e.g. the friction coefficient between the blood and the vessel wall. The simultaneous measurement of pressure and flow at a single or multiple locations may be used to calibrate the fluid dynamics model so that more accurate predictions may be made in other parts of the vascular system.

In a simple lumped model approach, a resistance R=k/A is attributed to every vessel segment. Where A is the cross sectional area of the segment and k is a proportionality constant which is influenced by e.g. the friction coefficient and the blood viscosity. From a measured flow Q and a pressure drop across a vessel segment, the proportionality constant k can be determined: k=A·Δp/Q.

In a further option, the width of the Doppler spectrum is measured as an additional parameter to improve the prediction capabilities in more complex implementations, e.g. if the angulation of the flow probe relative to the flow direction is an issue.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An intravascular flow determination device, comprising:
an input unit;
a data processing unit; and
an output unit;
wherein the input unit is configured to provide a local flow velocity value of a fluid measured with a flow sensor inside a vessel of an object, which local flow velocity value is measured at a local position of interest, and to provide local spatial data of the vessel and the local position of interest; wherein the local flow velocity value, and the local spatial data relate to the same position in time; and to provide a model flow-profile;
wherein the data processing unit is configured to adapt the model flow-profile based on the local values and the spatial data of the vessel and fluid dynamic constraints to generate an adapted local flow-profile relating to a cross-section at the local position of interest; and to determine a local peak flow value of the fluid inside the vessel based on the generated adapted local flow-profile; and
wherein the output unit is configured to provide the local peak flow value.

2. Device according to claim 1, wherein the input unit is further configured to provide a local pressure value of the fluid inside the vessel for the local position of interest; wherein the local pressure value relates to the same position in time; and wherein the data processing unit is configured to adapt the model flow-profile also based on the local pressure value.

3. Device according to claim 1, wherein the data processing unit is configured to output a ratio of two local peak flow velocities at two distinct locations.

4. A hemodynamic system for intravascular flow determination, comprising:
an X-ray imaging device;
a flow measure device comprising the flow sensor; and
an intravascular flow determination device according to claim 1;
wherein the flow measure device is configured to be arranged inside a vessel and to measure the local flow velocity value;
wherein the X-ray imaging device comprises an X-ray source and an X-ray detector to acquire image data of a region of interest of the vessel comprising a local position of interest;
wherein the data processing unit is configured to determine a position of the flow measure device arranged inside the vessel based on the acquired image data.

5. System according to claim 4, wherein it is further provided:
a pressure detection device;
wherein the pressure detection device is configured to detect a local pressure value; and
wherein the data processing unit is configured to determine a position of the pressure detection device arranged inside the vessel based on the acquired image data.

6. System according to claim 4, wherein, for adapting the model flow-profile, the data processing unit is configured:
i) to provide fluid dynamic constraints that comprise at least one of the following:
physiological data of the patient, such as age, weight, blood viscosity or other blood values, or a local pressure value;
vessel diameter derived from the spatial data;
vessel position derived from the spatial data;
relative position of the flow measure device within the vessel;
measured blood flow velocity at the position of the flow measure device; and
analytic equations based on a tube with a friction coefficient;
and/or
ii) to use a finite element fluid dynamics model as fluid dynamic constraints, wherein the finite element fluid dynamics model has as input parameters a local vessel geometry including a radius of the vessel, a relative position of the flow measure device within the vessel and the measured blood flow velocity at the position of the flow measure device, and, preferably, the measured local pressure value.

7. System according to claim 4, wherein the flow measure device is an ultrasound device; and
wherein, preferably, the flow is measured with Doppler ultrasound in a viewing direction.

8. A method for determining intravascular flow, comprising the following steps:
a) providing a local flow velocity value of a fluid measured with a flow sensor inside a vessel of an object, which local flow velocity value is measured at a local position of interest;
b) providing local spatial data of the vessel and the local position of interest;
wherein the local flow velocity value and the local spatial data relate to the same position in time;
c) providing a model flow-profile;
d) adapting the model flow-profile based on the local values and the spatial data of the vessel and fluid dynamic constraints to generate an adapted local flow-profile relating to a cross-section at the local position of interest;
e) determining, based on the generated adapted local flow-profile:
i) a local peak flow value of the fluid inside the vessel; and/or
ii) a local value for volumetric flow rate;
and
wherein, preferably, it is provided a step al) of providing a local pressure value of the fluid inside the vessel for the local position of interest; wherein the local pressure value relates to the same position in time; and wherein in step d) the adapting of the model flow-profile is also based on the local pressure values.

9. Method according to claim 8, wherein for a) it is provided:
measuring the local flow value at the local position of interest with the flow sensor of a flow measure device arranged inside the vessel; and
wherein for b) it is provided: measuring the local pressure value with a pressure device arranged inside the vessel; and
wherein for c) it is provided:
acquiring image data of a region of interest of the vessel comprising the local position of interest; and generating the local spatial data based on the image data; and
determining a position of the flow measure device arranged inside the vessel based on the acquired image data.

10. Method according to claim 8, wherein for c) it is provided:
generating at least one angiogram, for which at least one angiogram contrast agent injected X-ray images are acquired.

11. Method according to claim 8, wherein the fluid dynamic constraints comprise at least one of the following:
physiological data of the patient, such as age, weight, blood viscosity or other blood values, or a local pressure value;
vessel diameter derived from the spatial data;
vessel position derived from the spatial data;
relative position of the flow measure device within the vessel;
measured blood flow velocity at the position of the flow measure device; and
analytic equations based on a tube with a friction coefficient.

12. Method according to claim 8, wherein in e), the adapted local flow-profile is determined by using a finite element fluid dynamics model as fluid dynamic constraints, wherein the finite element fluid dynamics model has as input parameters a local vessel geometry including a radius of the vessel, a relative position of the flow measure device within the vessel and the measured blood flow velocity at the position of the flow measure device.

13. Method according to claim 8, wherein the ultrasound device has field of view and images an area displaced in the viewing direction;
wherein for the local spatial data, a position of the ultrasound device is detected; and wherein a displacement factor is applied for transforming the detected position of the ultrasound device into location data of the field of view in order to use the location data of the field of view as the local spatial data.

14. A computer program element for controlling an apparatus, which, when being executed by a processing unit, is adapted to perform the method steps of claim 8.

15. A computer readable medium having stored the program element of claim 14.
